(19) United States
(12) Patent Application Publication
(10) Pub. No.: US 2017/0296666 A1
(43) Pub. Date: Oct. 19, 2017

(54) Stable Pharmaceutical Composition Of Amorphous Ticagrelor

(71) Applicant: Amneal Pharmaceuticals Company GmbH, Steinhausen (CH)

(72) Inventors: Dhaval Thacker, Ahmedabad (IN); Atul Kathiriya, Ahmedabad (IN); Jatin Gajjar, Ahmedabad (IN)

(21) Appl. No.: 15/200,168

(22) Filed: Jul. 1, 2016

(30) Foreign Application Priority Data
Apr. 18, 2016 (IN) 201621013507

(51) Int. Cl. A61K 47/02; A61K 31/519

(52) U.S. Cl. CPC A61K 47/02; A61K 31/519

(57) ABSTRACT

A pharmaceutical composition comprises amorphous ticagrelor or a pharmaceutically acceptable salt thereof, a one or more pharmaceutically acceptable excipients, and colloidal silicon dioxide. Colloidal silicon dioxide is present in intragranular and in extragranular parts of the composition. Further, a pharmaceutical composition of amorphous ticagrelor or a pharmaceutically acceptable salt thereof is used in the treatment of a disease selected from the group consisting of myocardial infarction, thrombotic stroke, transient ischaemic attack, peripheral vascular disease and angina.

### CROSS-REFERENCE TO RELATED APPLICATIONS

This application claims priority to Indian Application Number 201621013507, filed on Apr. 18, 2016, which is incorporated herein by reference in its entirety.

### FIELD OF THE INVENTION

The present invention relates to a pharmaceutical composition comprising amorphous ticagrelor or a pharmaceutically acceptable salt thereof, a one or more pharmaceutically acceptable excipients, and colloidal silicon dioxide. The present invention further relates to a pharmaceutical composition of amorphous ticagrelor or a pharmaceutically acceptable salt thereof wherein colloidal silicon dioxide is present in intragranular and extragranular parts of the composition.

### BACKGROUND OF THE INVENTION

Ticagrelor is a P2Yn platelet aggregation inhibitor compound. Chemically, it is (1S,2S,3R,5S)-3-[7-{[(1/R,2S)-2-(3,4-difluorophenyl)cyclopropyl]amino}-5 (propylthio)-3H-[1,2,3]-triazolo[4,5-d]pyrimidin-3-yl]-5-(2-hydroxyethoxy) cyclopentane-1,2-diol. Ticagrelor is a crystalline powder with an aqueous solubility of approximately 10 pg/mL at room temperature.

According to the Biopharmaceutics Classification System (BCS), ticagrelor is classified as a class IV compound, exhibiting low solubility and low permeability. This property of ticagrelor leads to an undesirable dissolution profile from the formulation which effects on bioavailability. Further, this also leads to high intra-subject and inter-subject variability of formulation following oral administration.

Ticagrelor is marketed with brand name Brilinta. U.S. Pat. No. 6,251,910 discloses ticagrelor as one of the drug in P2T receptor antagonist class and U.S. Pat. No. 6,525,060 & U.S. Pat. No. 7,250,419 disclose specifically ticagrelor compound. Ticagrelor is approved in the treatment of acute coronary syndrome (unstable angina, non-ST elevation myocardial infarction, or ST elevation myocardial infarction) as disclosed by U.S. Pat. No. 6,525,060 & U.S. Pat. No. 7,250,419.

U.S. Pat. No. 8,425,934 discloses a pharmaceutical composition comprising ticagrelor, a filler consisting essentially of a mixture of mannitol and dibasic calcium phosphate dihydrate, a binder consisting essentially of hydroxypropyl cellulose, a disintegrant consisting essentially of sodium starch glycolate, and one or more lubricants. The compositions are prepared by using a wet granulation process.

WO 2015/001489 discloses a pharmaceutical composition comprising amorphous ticagrelor and one or more pharmaceutically acceptable excipients, wherein the pharmaceutical composition provides a desirable dissolution profile and enhanced bioavailability. It discloses that the dissolution of ticagrelor can be improved by using the drug in amorphous form.

WO 2014/170026 discloses a novel stabilized form of amorphous ticagrelor, a method of preparation of amorphous ticagrelor as well as pharmaceutical composition comprising the amorphous ticagrelor. It discloses that amorphous form of ticagrelor can be stabilized by mixing ticagrelor with a second component.

WO 2014/118808 discloses a novel amorphous solid dispersion of ticagrelor in combination with a pharmaceutically acceptable carrier. It further discloses a process for the preparation of amorphous solid dispersion of ticagrelor in combination with a pharmaceutically acceptable carrier wherein the solution of ticagrelor and one or more pharmaceutically acceptable carriers are prepared using a solvent and removing the solvent to obtain amorphous solid dispersion of ticagrelor.

US 2013/0028938 discloses a solid pharmaceutical dosage form comprising ticagrelor and a process of preparing the dosage form. It addresses the bioavailability problems associated with ticagrelor due to its poor solubility which can be improved by using certain particle size of ticagrelor in the formulation. It further discloses a solid pharmaceutical dosage form comprising particles of ticagrelor or a pharmaceutically acceptable salt or ester thereof, characterized in that at least 90% by volume of the ticagrelor particles have a particle size in the range of 1 μm to 150 μm.

It is known that amorphous form of ticagrelor poses more challenges during formulation preparation due to its physico-chemical properties. The present invention has observed the problem of using amorphous form of ticagrelor in formulation that when amorphous ticagrelor comes in contact with moisture, it forms a cohesive mass which prolongs the disintegration time as well as retards the dissolution of a formulation. Further, it is very difficult and cumbersome to prepare formulation which contains cohesive mass and to characterize it.

The aforementioned problems with respect to formation of cohesive mass in formulation by using amorphous form of ticagrelor are not addressed thus far. The present invention has addressed this problem by use of colloidal silicon dioxide in the formulation which inhibits the tendency of amorphous ticagrelor to form a cohesive mass. Specifically, the inventors have used colloidal silicon dioxide with a mixture of ticagrelor which inhibits the tendency of amorphous ticagrelor to form a cohesive mass. This is due to entrapment of colloidal silicon dioxide particles in between resulting amorphous particles of ticagrelor and thereby obviating the formulation obstacle and forms a stable formulation.

### SUMMARY OF THE INVENTION

The present invention relates to a pharmaceutical composition comprising an amorphous ticagrelor or a pharmaceutically acceptable salt thereof and a process of preparing the pharmaceutical composition.

The present invention specifically relates to a pharmaceutical composition comprising amorphous ticagrelor or a pharmaceutically acceptable salt thereof, a one or more pharmaceutically acceptable excipients, and colloidal silicon dioxide.

Further, the present invention relates to a pharmaceutical composition of amorphous ticagrelor or a pharmaceutically acceptable salt thereof, a one or more pharmaceutically acceptable excipients, and colloidal silicon dioxide wherein colloidal silicon dioxide is present in intragranular and extragranular parts of the composition.

### DETAILED DESCRIPTION

The present invention relates to a pharmaceutical composition comprising an amorphous ticagrelor or a pharmaceutically acceptable salt thereof and a process of preparing the pharmaceutical composition.

The term “Amorphous” refers a solid that lacks the long-range crystalline order, without definite shape or visible differentiation in structure.

The term “Intragranular” refers to being or occurring within granules of the composition i.e. granules comprising pharmaceutically acceptable active ingredient, a first pharmaceutically acceptable excipient component selected from the group consisting of a binder, a disintegrant, a diluent, a glidant and a solvent. All these elements fall under intragranular part of composition.

The term “Extra granular” refers to of or addition of pharmaceutically acceptable component to a material following granulation i.e a extra-granular fraction comprising a second pharmaceutically acceptable excipient component, wherein said second pharmaceutically acceptable excipient component selected from the group consisting of a disintegrant, a diluent, a lubricant, a glidant or the like.

The term “Cohesive mass” refers to a mass capable of adhering or sticking or having a tendency to unite and to resist separation especially in the presence of moisture.

The term “about” refers to any value which lies within the defined range by present inventors from a variation of up to ±10% of the claimed value.

A first aspect of the present invention relates to a pharmaceutical composition comprising amorphous ticagrelor or a pharmaceutically acceptable salt thereof, a one or more pharmaceutically acceptable excipients, and colloidal silicon dioxide.

The present invention addresses the problems associated with the use of amorphous form of ticagrelor in formulation that when amorphous ticagrelor comes in contact with moisture, it forms a cohesive mass which prolongs and delays the disintegration time of formulation as well as also retards the dissolution of a formulation. Further, it is very difficult and cumbersome to prepare formulation which contains cohesive mass and to characterize it.

The present invention has found an approach for solving this problem by use of colloidal silicon dioxide in the formulation which inhibits the tendency of amorphous ticagrelor to form a cohesive mass. The use of colloidal silicon dioxide to a mixture of solvent containing ticagrelor inhibits the tendency of amorphous ticagrelor to form a cohesive mass. Entrapment of colloidal silicon dioxide particles in between amorphous particles of ticagrelor leads to inhibition of cohesive mass formation in the formulation and improves the dissolution of formulation.

According to one embodiment of the present invention, there is provided a pharmaceutical composition of amorphous ticagrelor or a pharmaceutically acceptable salt thereof, wherein colloidal silicon dioxide present in intragranular and in extragranular parts of the composition.

Additionally, the pharmaceutical composition may contain colloidal silicon dioxide both in intragranular as well as in extragranular parts of the composition. Colloidal silicon dioxide may present in intragranular and extragranular parts of the composition in an amount from about 1% to about 30% by weight of the composition, preferably from about 3% to about 20% by weight of composition, more preferably from about 4% to about 10% by weight of composition.

The intragranular part of composition contains colloidal silicon dioxide in an amount from about 0.5% to about 20% by weight of the composition, preferably from about 4% to about 10% by weight of the composition. The intragranular part of a composition is prepared by granulation, slugging, or coating the inert core with a drug solution. Preferably, the inert core and drug solution both contains colloidal silicon dioxide as an excipient.

The extragranular part of the composition contains colloidal silicon dioxide in an amount from about 0.5% to about 10% by weight of the total composition, preferably from about 3% to about 8% by weight of the composition.

According to another embodiment of the present invention, there is provided a pharmaceutical composition of amorphous ticagrelor or a pharmaceutically acceptable salt thereof, wherein the amorphous form of ticagrelor or pharmaceutically acceptable salt thereof may present in amount from about 1% to about 40% by weight of composition, preferably from about 10 to about 35% by weight of composition and more preferably from about 20 to about 30% by weight of composition.

According to another embodiment of the present invention, there is provided a pharmaceutical composition of amorphous ticagrelor or a pharmaceutically acceptable salt thereof, a one or more pharmaceutically acceptable excipients, and colloidal silicon dioxide, wherein a one or more pharmaceutically acceptable excipients selected from diluents, binders, disintegrants, glidants, adsorbents, lubricants, and mixtures thereof.

The diluents used in the pharmaceutical composition of the present invention are selected from the group consisting of: an inorganic phosphates like dibasic calcium phosphate, or sugars or sugar analogues and derivatives thereof in particular lactose, such as lactose monohydrate or water-free lactose, dextrose, sorbitol, mannitol, saccharose, maltodextrin, isomalt, or celluloses like microcrystalline cellulose or powdered celluloses or the like. The diluents may present in an amount from about 10% to 80% by weight of composition, preferably from about 40% to 70% by weight of composition.

The binders used in the pharmaceutical composition of the present invention are selected from the group consisting of a polyvinylpyrrolidone (PVP), starch, cellulose derivatives like hydroxypropylmethyl cellulose, sucrose, lactose, xylitol, sorbitol, maltitol, water, alcohol or polyethelyne glycol or the like. According to the present invention, preferable binder are polyvinylpyrrolidone (plasdone k29/32), and polyethelyne glycol. The binders may present in an amount from about 1% to 10% by weight of composition, preferably from about 2% to 6% by weight of composition.

The disintegrants used in the pharmaceutical composition of the present invention are selected from the group consisting of a sodium starch glycolate, alginates, pregelatinized starch, croscarmellose and cross-linked PVP like collidone and crospovidone or the like. According to the present invention, preferable disintegrants are crospovidone and sodium starch glycolate. The disintegrants may present in an amount from about 1% to 10% by weight of composition, preferably from about 4% to 8% by weight of composition.

According to present invention, glidants present in the pharmaceutical dosage form are such as silicon dioxide, talc, magnesium stearate or the like. A preferred glidant is silicon dioxide and may present in amount from about 0.1% to 10% by weight of composition.

According to present invention, lubricants present in the pharmaceutical dosage form are such as fatty acids or fatty acid derivatives, such as alkali and earth alkali salts of stearic, lauric and/or palmitic acid. A preferred lubricant is magnesium stearate and may present in amount from about 0.1% to 10% by weight of composition.

According to another embodiment of the present invention, there is provided a pharmaceutical composition of amorphous ticagrelor or a pharmaceutically acceptable salt thereof wherein the ratio of intragranular components to extragranular components plays an important role in improving dissolution from composition. The intragranular components and extragranular components may present in a ratio from about 1:1 to about 40:1 preferably from about 4:1 to about 20:1.

The pharmaceutical composition of the present invention can be obtained by a known conventional methods like dry granulation, wet granulation, direct compression, roller compaction, fluidized bed granulation, rapid mixture granulation, solvent evaporation, hot-melt extrusion or the like.

According to another embodiment of the present invention, there is provided a pharmaceutical composition comprising from about 1% to about 40% w/w of ticagrelor or a pharmaceutically acceptable salt thereof, from about 1% to about 30% w/w of colloidal silicon dioxide, from about 10% to about 80% w/w of diluents, from about 1% to about 10% w/w of binders, from about 1% to about 10% w/w of disintegrants, from about 0.1% to about 10% w/w of lubricants, from about 0.1% to about 10% w/w of glidants and optionally from about 1% to about 10% w/w of film coating substance.

In particularly, the present invention provides a pharmaceutical composition comprising about 27% w/w of ticagrelor or a pharmaceutically acceptable salt thereof, about 49% w/w of microcrystalline cellulose, about 9% w/w of colloidal silicon dioxide, about 2.5% w/w of polyvinyl pyrrolidone, about 2.5% w/w of sodium starch glycolate, about 5% w/w of crospovidone, about 1% w/w of talc, about 1% w/w of magnesium stearate, and about 3% w/w of film coating material.

According to another aspect, the pharmaceutical composition is prepared by a process comprising the steps of:
preparing a dry mixture of one or more pharmaceutical excipients comprising colloidal silicon dioxide;
preparing a drug solution comprising ticagrelor or a pharmaceutically acceptable salt thereof, colloidal silicon dioxide and one or more pharmaceutical excipients; granulating the dry mixture prepared with the drug solution to form granules;
blending the granules obtained with extragranular excipients to form a blend;
compressing and/or filling the blend obtained to form a composition and optionally coating the composition.

A second aspect of the present invention provides a process for the preparation of a pharmaceutical composition of the present invention, wherein the process comprises the steps of:
blending amorphous ticagrelor, a one or more pharmaceutically acceptable excipients and colloidal silicon dioxide; further lubricating the blend; and directly compressing the lubricated blend into tablets or filling the lubricated blend into capsule dosage form.

A third aspect of the present invention provides a process for the preparation of the pharmaceutical composition of the present invention, wherein the process comprises the steps of:
blending amorphous ticagrelor, one or more diluents, binders, and disintegrants, and colloidal silicon dioxide;
compacting the blend to obtain granules or flakes;
lubricating the granules/flakes using the additional lubricants; and
compressing the lubricated granules into tablets or filling into capsules.

A fourth aspect of the present invention provides a process for the preparation of a pharmaceutical composition of the present invention, wherein the process comprises the steps of:
blending amorphous ticagrelor, one or more hydrophilic polymers, colloidal silicon dioxide and optionally a surfactant in a rapid mixer granulator;
loading the granules obtained into a hot melt extruder to form a solid dispersion in the form of extrudates;
milling the extrudates and adding one or more diluents, binders, disintegrants, and lubricants; and
compressing the granules into tablets or filling into capsules.

The composition of the present invention may be in the form of minitablets, granules, pellets, tablets, capsules or the like. The pharmaceutical composition of the present invention may further be film-coated using techniques well known in the art such as spray coating in a conventional coating pan or a fluidized bed processor or dip coating. Alternatively, coating may also be performed using the hot melt technique. The film coat comprises film-forming polymers, one or more pharmaceutically acceptable excipients and pharmaceutically acceptable solvents.

Examples of film-forming agents include, but are not limited to, cellulose derivatives such as methylcellulose, hydroxymethyl cellulose, hydroxyethyl cellulose, hydroxypropyl cellulose, hydroxymethyl ethylcellulose, hydroxypropyl methylcellulose, sodium carboxymethyl cellulose, and ethyl cellulose; waxes; fat substances; or mixtures thereof. Alternatively, commercially available coating compositions comprising film forming polymers marketed under various trade names, such as Opadry®, may be used for coating.

Examples of solvents used for preparing the coating solution are selected from methyl alcohol, ethyl alcohol, isopropyl alcohol, n-butyl alcohol, acetone, acetonitrile, chloroform, methylene chloride, water, or mixtures thereof.

The pharmaceutical composition of the present invention can be used in the treatment of a disease selected from the group consisting of myocardial infarction, thrombotic stroke, transient ischaemic attack, peripheral vascular disease and angina.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### Example 1

TABLE 1

| | | | | |
|---|---|---|---|---|
| Sr. | | | | |
| No. | Ingredients | Functionality | mg/tab | % w/w |

DRY MIX

| | | | | |
|---|---|---|---|---|
| 1 | Microcrystalline Cellulose | Diluent | 135.00 | 40.91 |
| | (Avicel PH102) | | | |
| 2 | Sodium Starch Glycolate | Disintegrant | 8.00 | 2.43 |
| | (Type A)(GLYCOLYS) | | | |
| 3 | Silicon Dioxide (Aeroperl 300 | Glident | 10.00 | 3.03 |
| | Pharma) | | | |

DRUG SOLUTION

| | | | | |
|---|---|---|---|---|
| 4 | Ticagrelor | Active | 90.00 | 27.27 |
| 5 | Povidone K-30 (Plasdone | Binder | 8.00 | 2.43 |
| | K29/32) | | | |
| 6 | Silicon Dioxide (Aeroperl 300 | Glidant | 10.00 | 3.03 |
| | Pharma) | | | |
| 7 | Ethanol (Absolute) | Solvent | QS | — |
| 8 | Dichloromethane | Solvent | QS | — |

EXTRAGRANULAR

| | | | | |
|---|---|---|---|---|
| 9 | Microcrystalline Cellulose | Diluent | 28.00 | 8.49 |
| | (Avicel PH102) | | | |
| 10 | Colloidal Silicon dioxide | Glidant | 10.00 | 3.03 |
| | (Aerosil 200) | | | |
| 11 | Crospovidone XL (Polyplasdone | Distintegrant | 15.00 | 4.55 |
| | XL) | | | |
| 12 | Talc (Micronized) | Glidant | 3.00 | 0.91 |
| 13 | Magnesium Stearate | Lubricant | 3.00 | 0.91 |
| | Core Total | | 320.00 | |

FILM COATING

| | | | | |
|---|---|---|---|---|
| 15 | Opadry Yellow 03B520162 | Coating | 10.00 | 3.03 |
| | | Material | | |
| | Total | | 330.00 | 100.00 |

Procedure:
1. Dispensed the material from 1 to 3 and was sifted through #40 ASTM.
2. Dissolve drug, povidone, silicon dioxide in a mixture of ethanol and dichloromethane solvent, wherein the drug dissolved completely.
3. The material of step 1 was pre-heated in FBP and granulated using Drug solution of step 2.
4. Dried the granulated mass till required % LOD is achieved.
5. Milled the granules through 30# ASTM OG.
6. Material from 9-11 was sifted through #30 ASTM, material 12 sifted through #60 ASTM and both of these materials were blended with dried milled granules of step-4 for 10 minutes in blender.
7. Lubricated the blend of step-5 with magnesium stearate sifted through mesh #60.
8. Compressed the blend of step 6 using suitable punches.
9. Coated the compressed tablets of step-7 with coating material.

### Comparative Example 1

TABLE 2

| | | | |
|---|---|---|---|
| Sr. | | | |
| No. | Ingredients | Mg/tab | % w/w |

DRUG SOLUTION

| | | | |
|---|---|---|---|
| 1 | Ticagrelor | 90.00 | 29.61 |
| 2 | PVP K 30 | 10.00 | 3.29 |
| 3 | Ethanol/DCM | q.s. | — |

INTRAGRANULAR

| | | | |
|---|---|---|---|
| 4 | Mannitol | 133.00 | 43.75 |
| 5 | MCC | 35.00 | 11.51 |
| 6 | Silicon Dioxide | 20.00 | 6.58 |

EXTRAGRANULAR

| | | | |
|---|---|---|---|
| 7 | SSG Type A | 8.00 | 2.63 |
| 8 | Talc | 3.00 | 0.99 |
| 9 | Magnesium Stearate | 5.00 | 1.64 |

| | | |
|---|---|---|
| CORE TOTAL | 304.00 | 100.00 |

Procedure
1. Dissolve drug, and binder in a mixture of ethanol and/or dichloromethane solvent, wherein the drug dissolved completely. Sprayed it on bed of Intra-granular material.
2. Milled through #24.
3. Blended & lubricated using SSG, Talc and Magnesium Stearate respectively.
4. Performed compression & coating of step 5 material.

### Comparative Example 2

TABLE 3

| | | | |
|---|---|---|---|
| Sr. | | | |
| No. | Ingredients | Mg/tab | % w/w |

DRUG SOLUTION

| | | | |
|---|---|---|---|
| 1 | Ticagrelor | 90.00 | 34.62 |
| 2 | PVP K 30 | 10.00 | 3.85 |
| 3 | MeOH/IPA | q.s. | — |

INTRAGRANULAR

| | | | |
|---|---|---|---|
| 4 | Mannitol | 147.00 | 5 |
| 6 | SSG Type A | 5.00 | 1.92 |

EXTRAGRANULAR

| | | | |
|---|---|---|---|
| 7 | SSG Type A | 5.00 | 1.92 |
| 8 | Magnesium Stearate | 3.00 | 1.15 |

| | | |
|---|---|---|
| CORE TOTAL | 260.00 | 100.00 |

Procedure:
1. Dissolve drug, and binder in a mixture of methanol and/or isopropyl alcohol solvent, wherein the drug dissolved completely.
2. Sprayed it on bed of Intra-granular material.
3. Milled through #24.
4. Blended & lubricated using SSG and Magnesium Stearate respectively.
5. Performed compression & coating of step 5 material.

All above mentioned examples described were characterized for disintegration and dissolution property behaviour of formulation according to pharmacopoeia reported method and results are described in below table 4.

TABLE 4

| | | Comparative | Comparative |
|---|---|---|---|
| | Example 1 | Example 1 | Example 2 |
| DISINTEGRATION | 2 min & 10 sec | 26 min | >30 min |
| TIME (Min) | | | |
| DISSOLUTION | In 900 ml water + 0.2% tween | | |
| PROFILE (Min) | 80 dissolution medium | | |
| 10 | 54 | 16 | Tablets forms a |
| 15 | 62 | 21 | gel as required |
| 30 | 91 | 31 | disintegration |
| 45 (80% Q POINT) | 98 | 38 | time is more and |
| | | | swells. |

As per the results mentioned in table 4, example 1 formulation meets the aspects of disintegration and dissolution profile of the present invention. While comparative example 1 formulation swells and disperse very slowly and further at 45 min time interval, only 38% of drug release occurs from the formulation. The comparative example 2 formulation swells and does not disintegrate and not found to be suitable as per the present invention.

What is claimed:

1. A pharmaceutical composition comprising amorphous ticagrelor or a pharmaceutically acceptable salt thereof, one or more pharmaceutically acceptable excipients, and colloidal silicon dioxide, wherein the colloidal silicon dioxide is present in intragranular and extragranular parts of the composition.

2. The pharmaceutical composition according to claim 1, wherein the pharmaceutically acceptable excipients are selected from the group consisting of: diluents, binders, disintegrants, glidants, adsorbents, lubricants, and mixtures thereof.

3. The pharmaceutical composition according to claim 1, wherein the amorphous form of ticagrelor or pharmaceutically acceptable salt thereof is present in amount from about 1% to about 40% by weight of composition.

4. The pharmaceutical composition according to claim 1, wherein the pharmaceutical composition present in the form of tablets, capsules, or pellets.

5. The pharmaceutical composition according to claim 1, wherein the colloidal silicon dioxide is present in the intragranular and extragranular parts in an amount from about 1% to about 30% by weight of composition.

6. The pharmaceutical composition according to claim 5, wherein the colloidal silicon dioxide is present in the intragranular and extragranular parts in an amount from about 3% to about 15% by weight of composition.

7. The pharmaceutical composition according to claim 1 comprising:
from about 1% to about 40% w/w of ticagrelor or a pharmaceutically acceptable salt thereof,
from about 1% to about 30% w/w of colloidal silicon dioxide,
from about 10% to about 80% w/w of diluents,
from about 1% to about 10% w/w of binders, from about 1% to about 10% w/w of disintegrants,
from about 0.1% to about 10% w/w of lubricants,
from about 0.1% to about 10% w/w of glidants, and
optionally from about 1% to about 10% w/w of a film coating substance.

8. The pharmaceutical composition according to claim 7, comprising:
about 27% w/w of ticagrelor or a pharmaceutically acceptable salt thereof,
about 49% w/w of microcrystalline cellulose,
about 9% w/w of colloidal silicon dioxide,
about 2.5% w/w of polyvinyl pyrrolidone,
about 2.5% w/w of sodium starch glycolate,
about 5% w/w of crospovidone,
about 1% w/w of talc,
about 1% w/w of magnesium stearate, and
about 3% w/w of film coating material.

9. The pharmaceutical composition according to claim 1, wherein the pharmaceutical composition is prepared by a process comprising:
preparing a dry mixture of one or more pharmaceutical excipients comprising colloidal silicon dioxide;
preparing a drug solution comprising ticagrelor or a pharmaceutically acceptable salt thereof, colloidal silicon dioxide and one or more pharmaceutical excipients;
granulating the dry mixture with the drug solution to form granules;
blending the granules with extragranular excipients to form a blend;
compressing or filling the blend to form a composition; and
optionally coating the composition.

10. The pharmaceutical composition according to claim 1, wherein the pharmaceutical composition is used in the treatment of a disease selected from the group consisting of: myocardial infarction, thrombotic stroke, transient ischaemic attack, peripheral vascular disease and angina.

11. A method of making a pharmaceutical composition comprises:
preparing a dry mixture of one or more pharmaceutical excipients comprising colloidal silicon dioxide;
preparing a drug solution comprising ticagrelor or a pharmaceutically acceptable salt thereof, colloidal silicon dioxide and one or more pharmaceutical excipients;
granulating the dry mixture with the drug solution to form granules;
blending the granules with extragranular excipients to form a blend;
compressing or filling the blend to form a composition; and
optionally coating the composition.
